SEQUENCE LISTING (13 sequences)

SEQ ID NO: 1 - DNA, 20 bp, synthetic construct
  source 1..20: mol_type = other DNA
tcaaagtcaagacagaaggg

SEQ ID NO: 2 - DNA, 20 bp, synthetic construct
  source 1..20: mol_type = other DNA
agggccacatgggacaaccg

SEQ ID NO: 3 - DNA, 20 bp, synthetic construct
  source 1..20: mol_type = other DNA
ccagctcactgttggtgaca

SEQ ID NO: 4 - DNA, 20 bp, synthetic construct
  source 1..20: mol_type = other DNA
gcccggggcagtcgaattga

SEQ ID NO: 5 - DNA, 20 bp, synthetic construct
  source 1..20: mol_type = other DNA
attctgccctctaaggctgt

SEQ ID NO: 6 - DNA, 20 bp, synthetic construct
  source 1..20: mol_type = other DNA
gagagcatcagagggttgtc

SEQ ID NO: 7 - DNA, 20 bp, synthetic construct
  source 1..20: mol_type = other DNA
gatgggggcccttgcctcag

SEQ ID NO: 8 - DNA, 20 bp, synthetic construct
  source 1..20: mol_type = other DNA
gataaagttctgagtttgag

SEQ ID NO: 9 - DNA, 20 bp, synthetic construct
  source 1..20: mol_type = other DNA
gccctcaattcgactgcccc

SEQ ID NO: 10 - DNA, 20 bp, synthetic construct
  source 1..20: mol_type = other DNA
tctgggctttacgctgacgc

SEQ ID NO: 11 - DNA, 20 bp, synthetic construct
  source 1..20: mol_type = other DNA
caagccctagatagccccag

SEQ ID NO: 12 - DNA, 20 bp, synthetic construct
  source 1..20: mol_type = other DNA
aagccctagatagccccaga

SEQ ID NO: 13 - DNA, 23 bp, synthetic construct
  source 1..23: mol_type = other DNA
ggtgacaagtgtgatcacttggg

(19) United States
(12) Patent Application Publication
(10) Pub. No.: US 2026/0224731 A1
(43) Pub. Date: Aug. 6, 2026

(54) Pharmaceutical Composition for Treating or Preventing Cancer Comprising Inhibitor of NUTM1 and Anticancer Agent as Active Ingredients and Use Thereof

(71) Applicants: Samsung Life Public Welfare Foundation, Seoul (KR); Research & Business Foundation Sungkyunkwan University, Suwon-si (KR)

(72) Inventors: Yoon-La Choi, Seoul (KR); Mi-Sook Lee, Seoul (KR); Juyoung Choi, Seoul (KR); Young Kee Shin, Seoul (KR); Sae Hyung Lee, Seoul (KR); Hee Geon Park, Seoul (KR)

(21) Appl. No.: 19/530,690

(22) Filed: Feb. 5, 2026

(30) Foreign Application Priority Data
Feb. 6, 2025 (KR) 10-2025-0015234

(51) Int. Cl. A61K 47/68; A61K 31/7088; A61K 38/21; A61K 38/46; A61K 39/395; A61P 35/00; C12N 15/11

(52) U.S. Cl. CPC A61K 47/6851; A61K 31/7088; A61K 38/215; A61K 38/465; A61K 39/3955; A61K 47/68037; A61K 47/6889; A61P 35/00; C12N 15/11; C12N 2310/20

(57) ABSTRACT

The present invention relates to a pharmaceutical composition for preventing or treating cancer, comprising an inhibitor of NUTM1 expression or activity and an anticancer agent as active ingredients, and a use thereof. More specifically, the present invention provides a therapeutic regimen for NUT carcinoma, which induces the expression of a TROP2 protein by specifically regulating NUTM1 gene expression and utilizes the same as a target for targeted therapy. That is, the NUTM1 inhibitor according to the present invention enhances the responsiveness to a TROP2-targeted anticancer agent, thereby increasing the anticancer effect when administered in combination with the TROP2-targeted anticancer agent. Accordingly, the present invention can enhance the therapeutic effect of the targeted anticancer agent in cancer patients, thereby suggesting a novel therapeutic strategy using the same.

### CROSS-REFERENCE TO RELATED PATENT APPLICATION

This application claims the benefit of Korean Patent Application No. 10-2025-0015234, filed on Feb. 6, 2025, in the Korean Intellectual Property Office, the disclosure of which is incorporated herein in its entirety by reference.

### REFERENCE TO AN ELECTRONIC SEQUENCE LISTING

The contents of the electronic sequence listing (JUNG-074_Sequence_Listing.xml; Size: 12,148 bytes; and Date of Creation: Apr. 8, 2026) is herein incorporated by reference in its entirety.

### BACKGROUND OF THE INVENTION

### 1. Field of the Invention

The present invention relates to a pharmaceutical composition for preventing or treating cancer, comprising an inhibitor of NUTM1 expression or activity and an anticancer agent as active ingredients, and a use thereof.

### 2. Description of the Related Art

Cancer treatment methods are broadly classified into three types: surgical therapy, anticancer drug therapy, and radiation therapy, and most anticancer agents currently used in anticancer drug therapy are cytotoxic anticancer agents. Although the development of cytotoxic anticancer agents has been steadily made, unfortunately, many cancer patients either do not respond to initial treatment or experience recurrence after an initial response to the treatment, ultimately resulting in death from progressive metastatic disease. Therefore, a continued focus on the design and discovery of novel anticancer agents remains very crucial.

Accordingly, although studies targeting mutant proteins that cause cancer progression are attracting attention, side effects sometimes occur because the strength of the mutual binding between biomarker proteins that exist specifically in cancer cells and anticancer agents is not specific. Moreover, although existing targeted therapeutics and immunotherapeutics have less toxicity and better effects than traditional compound anticancer agents, limitations in treatment are still being reported, and thus the development of therapeutics that specifically target cancer cells in such cancer treatment is urgently required. In particular, today's development of anticancer agents is being carried out based on the genome of cancer cells, enabling the treatment of patients who could not be treated previously due to the absence of patient-customized therapeutics, thereby contributing to efficient treatment, increased survival rates, and reduced side effects.

Among these, technology based on antibody-drug conjugates (ADCs), in which a cytotoxic drug is conjugated to an antibody that binds to an antigen expressed on the surface of cancer cells and capable of being internalized into the cells, is attracting attention for its ability to selectively deliver drugs to cancer cells, thereby accumulating drugs within cancer cells and killing them.

Meanwhile, nuclear protein in testis (NUT) carcinoma (NC), previously known as NUT midline carcinoma (NMC), is a fatal malignant tumor first reported in the literature in 1991, with a very low viability even with chemotherapy treatment. NUT carcinomas generally exhibit histological characteristics of an undifferentiated or poorly differentiated state, but in some cases, keratin pearl structures are formed due to abrupt keratinization, and the differentiated state shows a relatively good prognosis.

The genetic hallmark of NUT carcinoma is the rearrangement of NUT midline carcinoma family member 1 (NUTM1) gene with a series of partner genes, where an interchromosomal translocation between chromosomes 15q and 19p is mainly observed, creating a Bromodomain containing protein 4 (BRD4)-NUTM1 fusion oncogene. This BRD4-NUTM1 fusion oncoprotein induces overexpression of the oncoprotein Myc through chromatin remodeling, leading to a disruption of the gene expression regulatory network. To date, it is known that more than 70% of NUT carcinomas are predominantly caused by the BRD4-NUT fusion gene, and while BRD3 or NSD3 have been reported as partner genes of the NUT fusion gene, these are also bromodomain and extra terminal (BET) proteins, which belong to the same family as BRD4. Accordingly, the use of BET small molecular inhibitors that inhibit the function of BET has been proposed as a new treatment method for NUT carcinoma, but BET small molecular inhibitors have limitations in that they have low specificity for cancer genes, seriously affecting the expression of normal genes, resulting in significant side effects and development of drug resistance after a certain period of time.

Therefore, as a more effective approach for the prevention of cancer and the improvement of treatment, there is a need for a method capable of maximizing the therapeutic efficacy of cancer, based on various causative factors that act individually or collectively to initiate or promote the onset of cancer.

### PRIOR ART DOCUMENTS

### Patent Documents

Patent Document 1: KR 10-2351164 B1
Patent Document 2: KR 10-2731972 B1

### SUMMARY OF THE INVENTION

Accordingly, the present inventors have made extensive research efforts to develop a novel effective drug therapy for cancer, and as a result, have demonstrated that if cancer (e.g., NUT carcinoma) in a state refractory to conventional anticancer agents can be changed (e.g., differentiated) into a state responsive to conventional targeted anticancer therapy depending on the presence or absence (e.g., inhibition) of a therapeutic target (e.g., NUTM1) of the cancer, it is not only possible to maximize the cancer treatment efficacy by using a targeted anticancer agent in combination with an inhibitor that inhibits the expression level of the corresponding target gene, but also to implement personalized medicine, thereby completing the present invention.

Accordingly, an object of the present invention is to provide a pharmaceutical composition for preventing or treating cancer, comprising an NUTM1 inhibitor and a tumor-specific antigen-targeted anticancer agent as active ingredients.

Another object of the present invention is to provide a pharmaceutical composition for enhancing responsiveness to a tumor-specific antigen-targeted anticancer agent, comprising an NUTM1 inhibitor as an active ingredient.

Still another object of the present invention is to provide an adjuvant composition for a tumor-specific antigen-targeted anticancer agent, comprising an NUTM1 inhibitor as an active ingredient.

Yet another object of the present invention is to provide a health functional food for preventing or improving cancer, comprising an NUTM1 inhibitor and a tumor-specific antigen-targeted anticancer agent as active ingredients.

Still yet another object of the present invention is to provide a method for providing information required for the diagnosis of a patient exhibiting responsiveness to a tumor-specific antigen-targeted anticancer agent in a patient with NUT carcinoma.

The terms used in this specification are intended only for the purpose of illustration and should not be construed as limiting. Unless the context clearly indicates otherwise, the singular forms also include the plural forms. It should be understood that, as used herein, the terms “comprise”, “have”, or the like are intended to specify the presence of stated features, numerals, steps, operations, components, parts, or combinations thereof, but do not preclude the presence or addition of one or more other features, numerals, steps, operations, components, parts, or combinations thereof.

Unless otherwise defined, all terms used herein, including technical and scientific terms, have the same meaning as commonly understood by those skilled in the art to which the embodiments pertain. Terms, such as those defined in commonly used dictionaries, should be interpreted as having a meaning that is consistent with their meaning in the context of the relevant art, and will not be interpreted in an idealized or overly formal sense unless explicitly defined otherwise.

Hereinafter, the present invention will be described in detail.

According to an aspect of the present invention, the present invention provides a pharmaceutical composition for preventing or treating cancer, comprising an NUTM1 inhibitor and a tumor-specific antigen-targeted anticancer agent as active ingredients.

As used herein, the term “NUTM1 (NUT midline carcinoma family member 1)” refers to the NUTM1 gene on chromosome 15, also known as FAM22H or C15orf55, and is known to encode the genetic information of NUT (Nuclear protein in testis, NUT family member 1), which is a nuclear protein primarily expressed in testicular tissue.

As the NUTM1 inhibitor of the present invention, any inhibitor known in the art may be used as long as the object of the present invention can be achieved, and for example, it may be at least one selected from the group consisting of a CRISPR/Cas system comprising a guide RNA (gRNA) targeting the NUTM1 gene and a Cas protein (CRISPR associated protein), an antisense oligonucleotide (ASO), a small interference RNA (siRNA), a short hairpin RNA (shRNA), a microRNA (miRNA), and a ribozyme, and may preferably be a CRISPR/Cas system comprising a guide RNA targeting the NUTM1 gene and a Cas protein, but is not limited thereto.

Moreover, the NUTM1 inhibitor may be at least one selected from the group consisting of a compound, a peptide, a peptidomimetic, a substrate analog, an aptamer, and an antibody, each of which specifically binds to the NUT protein, and is not limited thereto as long as the object of the present invention can be achieved.

Furthermore, the guide RNA targeting the NUTM1 gene may be a guide RNA consisting of a nucleotide sequence selected from the group consisting of SEQ ID NOs: 1 to 12.

According to one embodiment of the present invention, the guide RNA consisting of a nucleotide sequence selected from the group consisting of SEQ ID NOs: 1 to 9 may target different sequences in the exon 3 region of the NUTM1 gene.

According to one embodiment of the present invention, the guide RNA consisting of the nucleotide sequence of SEQ ID NO: 10 may target a partial sequence of the exon 6 region of the NUTM1 gene.

According to one embodiment of the present invention, the guide RNA consisting of a nucleotide sequence selected from the group consisting of SEQ ID NOs: 11 to 12 may target different sequences in the exon 8 region of the NUTM1 gene.

In the present invention, the guide RNA may be a dual guide RNA consisting of a CRISPR RNA (crRNA) and a trans-activating crRNA (tracrRNA), or a single-stranded guide RNA (sgRNA) in which the crRNA and tracrRNA are linked to each other. Preferably, it may be a single-stranded guide RNA (sgRNA).

Meanwhile, the present invention may provide a recombinant guide RNA vector for targeting the NUTM1 gene, comprising a sequence encoding the guide RNA.

As used herein, the term “vector” refers to DNA that can be propagated by introducing a desired DNA fragment into a host cell, and is also called a cloning vehicle. The term “expression vector” refers to a recombinant DNA molecule comprising a desired coding sequence and appropriate nucleic acid sequences essential for expressing the operably-linked coding sequence in a specific host organism. In the present invention, the term “vector” may be used in the same sense as an expression vector.

In the present invention, the vector may be an expression vector in another form, which provides an equivalent function, such as a virus-like particle (VLP), a lentiviral vector, an adeno-associated virus (AAV) vector, an adenoviral vector, a herpesvirus vector, a retroviral vector, a vaccinia virus vector, a poxvirus vector, and a herpes simplex virus vector, and preferably, it may be a virus-like particle (VLP).

In addition, the present invention may provide a CRISPR/Cas9 complex for inhibiting NUTM1 gene expression, comprising the guide RNA vector and a Cas9 nuclease.

In the present invention, “Cas9” refers to “CRISPR-Cas9”, and CRISPR-Cas9 is the third-generation genetic scissors that recognizes, cleaves, and edits a specific nucleotide sequence to be used, and is useful for simply, rapidly, and efficiently performing manipulations such as inserting a specific gene into a target site of the genome or inactivating the activity of a specific gene.

In the present invention, the Cas9 protein refers to an essential protein component in the CRISPR/Cas system, and forms an active endonuclease or a nickase when forming a complex with two RNAs called crRNA and tracrRNA.

Information on the Cas9 protein or gene may be obtained from publicly available databases, such as GenBank of the National Center for Biotechnology Information (NCBI), but is not limited thereto. Moreover, the Cas9 protein may be appropriately linked to additional domains by those skilled in the art depending on the intended purpose.

In the present invention, the Cas9 protein may include not only wild-type Cas9 but also any variant of Cas9 as long as it has the function of a nuclease for gene editing. In the present invention, the origin of the Cas9 protein is not limited, and as a non-limiting example, it may be derived from a bacterial species selected from the group consisting of Streptococcus pyogenes, Streptococcus thermophilus, Streptococcus sp., Streptomyces pristinaespiralis, Streptomyces viridochromogenes, Streptosporangium roseum, Campylobacter jejuni, and Staphylococcus Auricularis, and may be appropriately selected and used by those skilled in the art. In one embodiment of the present invention, a Cas9 protein (spCas9) derived from Streptococcus pyogenes was used, but is not limited thereto.

As used herein, the term “inhibition of gene expression” refers to any activity that reduces the expression of a gene, and specifically, may be achieved by gene knock-out, knock-down, or introducing mutations such as deletion, duplication, inversion, or replacement into a gene DNA sequence.

The present invention may induce a modification in a target nucleic acid sequence by contacting the CRISPR/Cas9 complex comprising the guide RNA, which targets the NUTM1 gene, and the Cas9 protein, with the target nucleic acid in which the NUTM1 gene is encoded, and the modification may be a deletion, insertion, substitution, or insertion and deletion (indel) of at least one nucleotide compared to the wild-type NUTM1 gene sequence, or a cleavage of the target nucleic acid.

Transduction of the guide RNA and Cas9 protein into cells may be performed by directly introducing a pre-assembled complex (ribonucleoprotein) of the guide RNA and Cas9 protein into immune cells by a conventional method (e.g., electroporation, lipofection, etc.), by introducing a DNA molecule encoding the guide RNA and a gene (DNA or mRNA) encoding the Cas9 protein (or a gene having 80% or more, 85% or more, 90% or more, 95% or more, 96% or more, 97% or more, 98% or more, or 99% or more sequence homology therewith) into cells in a state of being contained in a single vector or separate vectors (e.g., a plasmid, a viral vector, etc.), or via mRNA delivery. In one embodiment of the present invention, the nucleic acid encoding the Cas9 protein may be contained in a virus-like particle (VLP).

The Cas9 protein, the guide RNA, the ribonucleoprotein (RNP) comprising the same, or the vector comprising a nucleic acid encoding at least one gene thereof may be delivered in vivo or into cells through an appropriate method selected from various methods known in the art, such as electroporation, lipofection, viral vectors, and nanoparticles, as well as protein translocation domain (PTD) fusion protein methods.

Furthermore, the NUTM1 inhibitor of the present invention may increase the expression level or activity of at least one tumor-specific antigen selected from the group consisting of trophoblast cell surface antigen 2 (Trop2), epidermal growth factor receptor (EGFR), vascular endothelial growth factor receptor (VEGFR), CD20, CD38, RANK-L, BTK, Bcr-abl, PDGFR/FGFR families, MEK/RAF, HER2/Neu, ubiquitin, JAK, MAP2K, ALK, PARP, transforming growth factor β (TGFβ) receptor, proteasome, Bcl-2, C-Met, VR1, VR2, VR3, c-kit, AXL, RET, Braf, DNA methyltransferase (DNMT), CDK4/6, and STING, and preferably, may increase the expression level or activity of Trop2.

In addition, the tumor-specific antigen-targeted anticancer agent may be a targeted anticancer agent that targets at least one tumor-specific antigen selected from the group consisting of Trop2, EGFR, VEGFR, CD20, CD38, RANK-L, BTK, Bcr-abl, PDGFR/FGFR families, MEK/RAF, HER2/Neu, ubiquitin, JAK, MAP2K, ALK, PARP, TGFβ receptor, proteasome, Bcl-2, C-Met, VR1, VR2, VR3, c-kit, AXL, RET, Braf, DNMT, CDK4/6, and STING, and preferably, targets Trop2.

In this regard, it has been identified that the guide RNA according to the present invention specifically binds to the NUTM1 gene, causing gene editing in the NUTM1 gene through the CRISPR/Cas system, and ultimately inhibiting the expression of the BRD4-NUTM1 fusion gene, thereby inhibiting the proliferation of NUT cancer cells and exhibiting anticancer effects such as apoptosis (see KR 10-2731972 B1). However, interestingly, it was first discovered in the present invention that when the expression level of NUTM1 is inhibited with an NUTM1 inhibitor, a tumor-specific antigen, Trop2, increases according to the differentiation of NUT carcinoma, despite the fact that the expression level of tumor-specific antigens should decrease according to cancer cell death as conventionally known in the art.

That is, the present invention confirmed an increase in Trop2 expression in NUT carcinoma when NUTM1 gene expression was inhibited by NUTM1 gene editing, and further, as a result of analyzing data from NUT cancer patients, the present invention first discovered that patients exhibiting a high degree of NUT carcinoma differentiation and TROP2 positivity are associated with a relatively better prognosis. Therefore, the characteristic feature of the present invention is that excellent activity for preventing or treating cancer is exhibited as a synergistic and complementary effect occurs through the combined use of an NUTM1 inhibitor and a TROP2-targeted anticancer agent.

Accordingly, the present invention established a strategy of recommending a TROP2-targeted therapeutic agent for NUT cancer patients with a high degree of differentiation and TROP2-positivity, and inducing cancer cell differentiation and TROP2 expression through NUTM1 gene editing for NUT cancer patients with a low degree of differentiation and TROP2-negativity to enable subsequent TROP2-targeted therapy; and further demonstrated that excellent desired effects are achieved by treating with a TROP2-targeted anticancer agent, which is a therapeutic agent targeting Trop2, through the strategy as described above.

In the present invention, the anticancer agent may be used without limitation as long as it is a drug effective in treating cancer, and may be preferably a TROP2-targeted anticancer agent. As the TROP2-targeted anticancer agent, any anticancer agent known in the art that targets Trop2 may be used as long as it can achieve the object of the present invention, and preferably, may be at least one selected from the group consisting of an anti-Trop2 antibody specifically binding to a Trop2 protein, an anti-Trop2 antibody-drug conjugate (ADC), an anti-Trop2 antibody-cytokine fusion protein (ACFP, immunocytokine), a peptide, a peptidomimetic, a substrate analog, an aptamer, and a compound. For example, the TROP2-targeted anticancer agent may be at least one selected from the group consisting of sacituzumab, a sacituzumab-IFN-β mutein fusion protein, sacituzumab govitecan (Trodelvy), datopotamab deruxtecan (DS-1062), ESG-401, SKB-264, DAC-02, and BAT-8003, but is not limited thereto.

In one embodiment of the present invention, sacituzumab, which is an anti-Trop2 antibody specifically binding to the Trop2 protein, a sacituzumab-IFN-β mutein fusion protein, which is an ACFP, and Sacituzumab Govitecan, which is an ADC, were used as the TROP2-targeted anticancer agent.

As used herein, the term “prevention” refers to any activity that inhibits or delays the onset of cancer by administration of the pharmaceutical composition. The term “treatment” refers to any activity that improves or advantageously changes the symptoms of cancer by administration of the pharmaceutical composition.

In the present invention, the cancer may be a BRD4-NUTM1-positive solid tumor, and may include squamous cell carcinoma or NUT carcinoma that mainly occurs in the upper tissues of the body, such as the thymus, airway, and lungs. It may preferably be lung cancer, thymic cancer, renal cancer, or NUT carcinoma. The NUT carcinoma is not limited to a specific tissue and may occur in all tissues.

Additionally, according to a preferred embodiment of the present invention, the NUT carcinoma may be in an undifferentiated or differentiated state.

In the present invention, the pharmaceutical composition may be used in a method for preventing or treating cancer, and specifically, the method for preventing or treating cancer may include administering the composition to a subject who has developed cancer or is expected to develop cancer.

As used herein, the term “administration” refers to introducing the composition to a subject by an appropriate method.

The composition of the present invention may be administered to a patient as a target subject in a pharmaceutically effective amount, and the patient to be administered may be a mammal, preferably a human, a monkey, or a rodent (mouse or rat), and in particular, may be any mammal, such as a human, that has a disease or symptom associated with the expression of a BRD4-NUTM1 fusion gene, or has the BRD4-NUTM1 fusion gene and is in need of inhibition of the expression of BRD4-NUTM1 and/or NUTM1.

The “pharmaceutically effective amount” refers to an amount capable of exhibiting a desired effect, that is, a gene-editing effect, at an application site, and may be variously prescribed depending on factors such as a formulation method, an administration mode, a patient's age, weight, sex, pathological condition, administration time, administration route, excretion rate, and response sensitivity.

The composition of the present invention may be prepared by further including at least one pharmaceutically acceptable carrier in addition to the above-described active ingredients. The pharmaceutically acceptable carrier must be compatible with the active ingredients of the present invention, and may be mixed with one or more of saline, sterile water, Ringer's solution, buffered saline, dextrose solution, maltodextrin solution, glycerol, ethanol, or mixtures thereof. Moreover, it may further comprise other conventional additives such as antioxidants, buffers, and bacteriostats as necessary. Furthermore, the composition may be formulated into an injectable formulation such as an aqueous solution, a suspension, or an emulsion by additionally adding a diluent, a dispersant, a surfactant, a binder, and a lubricant. In addition, the composition may be preferably formulated according to each disease or ingredient using an appropriate method in the art or a method disclosed in Remington's Pharmaceutical Science (Mack Publishing Company, Easton PA).

The content of the active ingredients and the like included in the composition of the present invention and an administration mode may be determined by those skilled in the art based on the symptoms and severity of disease of a patient. Moreover, the composition may be formulated in various forms, such as powders, tablets, capsules, solutions, injections, ointments, and syrups, and may also be provided in unit-dose or multi-dose containers, such as sealed ampoules and vials.

The composition of the present invention may be administered orally or parenterally. The administration routes of the composition of the present invention may include, but are not limited to, oral, intravenous, intramuscular, intra-arterial, intramedullary, intrathecal, intracardiac, transdermal, subcutaneous, intraperitoneal, enteral, sublingual, or topical administration. The dosage of the composition according to the present invention may vary depending on the patient's weight, age, sex, health condition, diet, administration time, administration route, excretion rate, or disease severity, and may be easily determined by those skilled in the art. Furthermore, the composition of the present invention may be formulated into a suitable dosage form for clinical administration using known techniques.

The present invention provides a pharmaceutical composition for preventing or treating cancer, comprising an NUTM1 inhibitor, which is administered together with an effective amount of a tumor-specific antigen-targeted anticancer agent.

In one embodiment of the present invention, the tumor-specific antigen-targeted anticancer agent may be administered simultaneously, separately, or sequentially with the NUTM1 inhibitor, and the effective amount may vary depending on a formulation method, an administration mode, an administration time, and/or an administration route of the pharmaceutical composition, and those skilled in the art may easily determine and prescribe an effective dosage for a desired treatment.

According to another aspect of the present invention, the present invention provides a pharmaceutical composition for enhancing responsiveness to a tumor-specific antigen-targeted anticancer agent, comprising an NUTM1 inhibitor as an active ingredient. Specifically, when treated in combination with cancer cells, the pharmaceutical composition enhances responsiveness to the tumor-specific antigen-targeted anticancer agent, thereby lowering the dosage of the anticancer agent used and improving the side effects of the anticancer agent.

According to still another aspect of the present invention, the present invention provides an adjuvant composition for a tumor-specific antigen-targeted anticancer agent, comprising the above-described NUTM1 inhibitor as an active ingredient.

As used herein, the term “anticancer adjuvant” refers to an agent that can be used as an adjuvant to enhance the effect of an anticancer therapy commonly used in the art. The composition may be in the form of a pharmaceutical composition or a food composition.

That is, in the present invention, it refers to an agent that can be administered in combination with an anticancer agent to enhance the responsiveness (sensitivity) to the anticancer agent, thereby improving, enhancing, or increasing the anticancer effect.

The adjuvant of the present invention may be administered simultaneously, separately, or sequentially with the anticancer agent. The administration order of the anticancer adjuvant according to the present invention, i.e., whether to administer the anticancer agent and the anticancer adjuvant simultaneously, separately, or sequentially, and at what point in time, may be determined by those skilled in the art. This administration order may vary depending on many factors. The anticancer adjuvant may be administered in combination with a known compound that has the effect of preventing, improving, or treating cancer. In this case, it may be administered simultaneously or sequentially with the known compound.

The anticancer adjuvant composition according to the present invention can maximize therapeutic effects and reduce side effects by increasing the sensitivity to the targeted anticancer agent through the combined use of an NUTM1 inhibitor. Furthermore, it can maximize anticancer effects by simultaneously blocking multiple pathways of cancer in combination with a CRISPR/Cas system comprising a guide RNA that inhibits the expression of various growth factors (e.g., VEGF, EGF, PDGF, etc.), growth factor receptors and downstream signaling proteins, viral oncogenic factors, and anticancer drug resistance genes.

According to yet another aspect of the present invention, the present invention provides a food composition for preventing or improving cancer, comprising the above-described NUTM1 inhibitor and tumor-specific antigen-targeted anticancer agent as active ingredients, or a health functional food comprising the same.

As used herein, the term “food” refers to a natural product or a processed product containing one or more nutrients, preferably a product that has become ready-to-eat through a certain degree of processing, and in a conventional sense, includes all of foods, food additives, health functional foods, and beverages.

The food composition of the present invention can be used as a health functional food. The term “health functional food” refers to a food manufactured and processed using raw materials or ingredients having functionality useful for the human body according to the Health Functional Food Act, and the term “functionality” refers to ingestion for the purpose of obtaining beneficial effects for health purposes, such as regulating nutrients or physiological functions for the structure and functions of the human body.

The food composition of the present invention may include conventional food additives, and unless otherwise specified, its suitability as a “food additive” is determined according to the specifications and standards for the relevant item in accordance with the General Rules and General Test Methods of the Food Additive Code approved by the Ministry of Food and Drug Safety.

In addition, the composition of the present invention may be used as a food supplement additive for health supplement foods for the prevention or improvement of cancer. Foods to which the composition of the present invention may be added include various foods, such as beverages, gums, teas, vitamin complexes, and health supplement foods, and the composition may be used in the form of pills, powders, granules, infusions, tablets, capsules, or beverages.

In the present invention, the food has bioregulatory functions such as prevention and improvement of cancer, biological defense, immunity, and recovery after illness, and it must be harmless to the human body when ingested for a long period of time.

When the food composition of the present invention is a food additive, the active ingredients may be added as they are or may be used together with other foods or food ingredients, and may be appropriately used according to a conventional method. The mixing amount of the active ingredients may be appropriately determined depending on the purpose of use (prevention, health, or therapeutic treatment). Generally, in the manufacture of a food or beverage, the active ingredients of the present invention may be added in an amount of 15% by weight or less, preferably 10% by weight or less, based on the raw materials. However, in the case of long-term ingestion for the purpose of health and hygiene or for health regulation, the amount may be equal to or less than the above range, and since there is no problem in terms of safety, the active ingredients may also be used in an amount equal to or greater than the above range.

There are no specific limitations on the types of the food. Examples of foods to which the active ingredients may be added include meat, sausages, bread, chocolate, candies, snacks, confectionery, pizza, ramen, other noodles, gums, dairy products including ice cream, various soups, beverages, teas, health drinks, alcoholic beverages, and vitamin complexes, and include all health functional foods in a conventional sense.

The health functional food composition according to the present invention may be in various forms, such as a health beverage. If the health functional food composition of the present invention is in the form of a health drink, it may include various flavoring agents or natural carbohydrates as additional ingredients, like conventional beverages. The natural carbohydrates described above may include monosaccharides such as glucose and fructose, disaccharides such as maltose and sucrose, natural sweeteners such as dextrin and cyclodextrin, or synthetic sweeteners such as saccharin and aspartame. The ratio of the natural carbohydrate is generally about 0.01 to 10 g, preferably about 0.01 to 0.1 g, per 100 ml of the composition of the present invention.

In addition to the above, the food composition or health functional food composition of the present invention may include various nutrients, vitamins, electrolytes, flavoring agents, coloring agents, pectic acid and its salts, alginic acid and its salts, organic acids, protective colloid thickeners, pH regulators, stabilizers, preservatives, glycerin, alcohol, and carbonating agents used in carbonated beverages. Additionally, the composition of the present invention may include fruit flesh for the manufacture of natural fruit juices, fruit juice beverages, and vegetable beverages. These ingredients may be used independently or in combination. The ratio of these additives is not particularly critical, but is typically selected in the range of 0.01 to 0.1 parts by weight per 100 parts by weight of the composition of the present invention.

According to still yet another aspect of the present invention, the present invention provides an anticancer feed composition or an animal feed additive comprising the above-described NUTM1 inhibitor and tumor-specific antigen-targeted anticancer agent as active ingredients.

The feed composition or feed additive of the present invention may comprise the above-described active ingredients of the present invention to exhibit an anticancer effect, and thus, when used as a feed additive, it can contribute to the enhancement of the health of target livestock.

The feed composition or feed additive of the present invention may include the active ingredients of the present invention as they are, or may further include known carriers and stabilizers such as grains and their by-products acceptable for livestock. If necessary, the feed composition or feed additive may further include organic acids such as citric acid, fumaric acid, adipic acid, lactic acid, and malic acid; phosphates such as sodium phosphate, potassium phosphate, acid pyrophosphate, and polyphosphate; natural antioxidants such as polyphenol, catechin, alpha-tocopherol, rosemary extract, vitamin C, green tea extract, licorice extract, chitosan, tannic acid, and phytic acid; antibiotics; antimicrobial agents; and other additives. The feed composition or feed additive may be in an appropriate form such as powder, granules, pellets, or suspension, and the feed composition or feed additive may be supplied to livestock alone or mixed with feed.

Since the composition of the present invention includes the above-described active ingredients of the present invention, redundant descriptions are omitted to avoid excessive complexity of the present specification.

According to a further aspect of the present invention, the present invention provides a method for providing information required for the diagnosis of a patient exhibiting responsiveness to a tumor-specific antigen-targeted anticancer agent in a patient with NUT carcinoma, the method comprising measuring the expression level of an NUTM1 gene or a protein thereof in a tumor tissue sample isolated from the patient with NUT carcinoma.

For example, the method of the present invention is intended to provide information for determining a response to a therapeutic agent targeting TROP2 in a patient with NUT carcinoma in which the expression of TROP2 is increased by treatment with a CRISPR/Cas system comprising an NUTM1-targeting gRNA. The method of the present invention may include providing information on whether the patient does not exhibit resistance to a TROP2-targeted anticancer agent, whether the agent can effectively inhibit the progression of cancer, or whether the agent can kill cancer cells, and is not limited thereto as long as it can obtain information related to the cancer treatment effect of the TROP2-targeted anticancer agent from a biological sample.

Regarding the method of the present invention, redundant descriptions are omitted to avoid excessive complexity of the present specification.

Since the NUTM1 inhibitor according to the present invention enhances the responsiveness to a targeted anticancer agent, thereby increasing the anticancer effect when administered in combination with the targeted anticancer agent, the present invention can increase the treatment success rate of cancer patients for the targeted anticancer agent, thereby suggesting new possibilities for a therapeutic strategy using the same.

### BRIEF DESCRIPTION OF THE DRAWINGS

FIG. 1 is a schematic diagram of a therapeutic strategy for NUTM1-expressing NUT carcinoma according to the present invention, illustrating the process of editing an NUTM1 gene through a CRISPR/Cas9 system in NUT carcinoma cells expressing an NUTM1 fusion gene, thereby inducing the expression of a TROP2 protein, and then treating with an anti-Trop2 antibody-targeted anticancer agent. The anti-TROP2 antibody-targeted anticancer agent binds to NUT carcinoma cells expressing TROP2 to induce a direct cytotoxic effect or inhibit the growth of cancer cells through an immunomodulatory mechanism. In particular, it can exhibit cancer cell-specific cytotoxicity induced by the targeted anticancer agent and antibody-dependent cellular cytotoxicity (ADCC) effects.

FIGS. 2A-2C illustrate the changes in cell morphology caused by NUTM1 gene editing and the results of differentially expressed gene (DEG) analysis.

FIGS. 3A-3D illustrate the increased expression of TROP2 in NUTM1-edited NUT carcinoma cell lines.

FIGS. 4A-4E illustrate the synergistic antitumor effect of an NUTM1 inhibitor and Trop2-targeting ACFP (sacituzumab-IFN-β mutein fusion protein) and ADC (sacituzumab govitecan) in NUT carcinoma cell lines.

FIGS. 5A-5E illustrate the expression of Trop2 in patient tissues according to the degree of NC differentiation.

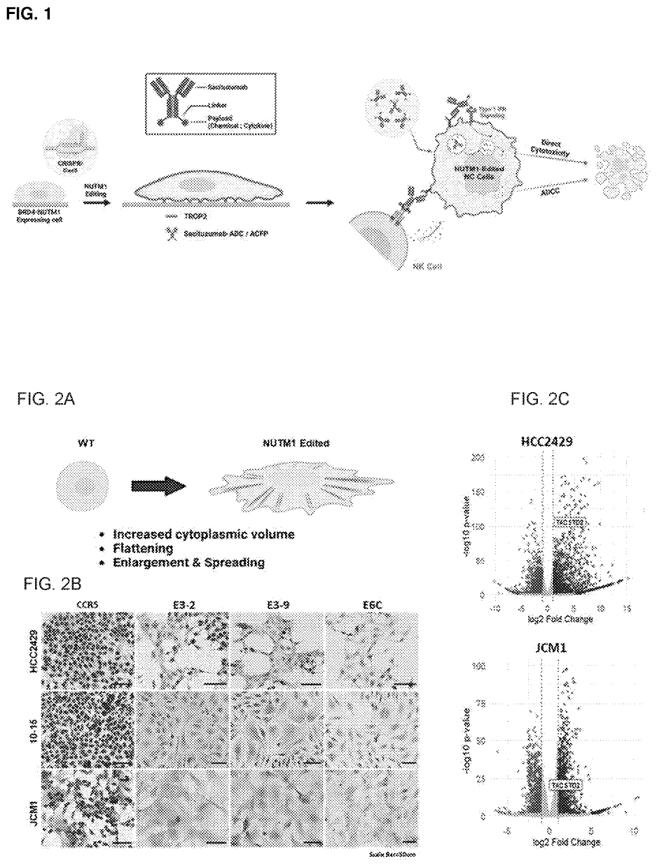

### DETAILED DESCRIPTION OF THE INVENTION

Hereinafter, the present invention will be described in detail with reference to Examples. However, the following Examples are only for illustrating the present invention, and the content of the present invention is not limited to the following Examples.

### Example 1: Design of Single Guide RNA of CRISPR/Cas9 System for Targeting NUTM1

As disclosed in Korean Patent No. 10-2731972, in order to inhibit the expression of a BRD4-NUTM1 fusion gene, the present inventors designed sgRNAs targeting the exon 3 of the NUTM1 gene (Genbank Accession number NM_001284292.2) and the p300 binding regions (the exons 6 and 8 of the NUTM1 gene) on the NUTM1 gene as the breakpoints, i.e., target sites, and secured a total of 12 candidate sequences of sgRNAs targeting the NUTM1 gene (Table 1). Among these, sg E3-2 (E3-2) (SEQ ID NO: 2), sg E3-9 (E3-9) (SEQ ID NO: 9), and E6C (a mixture of sg E6 (SEQ ID NO: 10) and sg E8-1v2 (SEQ ID NO: 12) in a ratio of 1:1 in the form of eVLPs), which were proven to be capable of inhibiting the function of the NUTM1 gene by efficiently cleaving the target DNA sequence and which exhibited excellent DNA cleavage efficiency, were selected and utilized in the following Examples.

TABLE 1

| | | | Partial sequence | |
|---|---|---|---|---|
| | | | of sgRNA | |
| | | | complementary | |
| | | | to target | PAM |
| SEQ | | | sequence | Se- |
| ID | Target | sgRNA | (indicated as DNA) | quence |
| NO | Exon | Name | (5′→3′) | (5′→3′) |
| 1 | 3 | sg E3-1 | TCAAAGTCAAGACAGAAGGG | GGG |
| 2 | 3 | sg E3-2 | AGGGCCACATGGGACAACCG | GGG |
| 3 | 3 | sg E3-3 | CCAGCTCACTGTTGGTGACA | GGG |
| 4 | 3 | sg E3-4 | GCCCGGGGCAGTCGAATTGA | GGG |
| 5 | 3 | sg E3-5 | ATTCTGCCCTCTAAGGCTGT | TGG |
| 6 | 3 | sg E3-6 | GAGAGCATCAGAGGGTTGTC | TGG |
| 7 | 3 | sg E3-7 | GATGGGGGCCCTTGCCTCAG | TGG |
| 8 | 3 | sg E3-8 | GATAAAGTTCTGAGTTTGAG | AGG |
| 9 | 3 | sg E3-9 | GCCCTCAATTCGACTGCCCC | GGG |
| 10 | 6 | sg E6 | TCTGGGCTTTACGCTGACGC | CGG |
| 11 | 8 | sg E8-1 | CAAGCCCTAGATAGCCCCAG | AGG |
| 12 | 8 | sg E8-1v2 | AAGCCCTAGATAGCCCCAGA | GGG |

### Example 2: Preparation of Engineered Virus-Like Particles (eVLPs)

Cas9-eVLPs were produced by transient transfection of Lenti-X 293T cells (632180, Clontech). Lenti-X 293T cells were cultured by seeding 1×107 cells in a 150 mm dish in DMEM medium (20 ml, SH30243.01, Hyclone) supplemented with 10% (v/v) FBS (16000-044, Gibco). After 24 hours, VSV-G (800 ng; 12259, Addgene), gag-pol (6,750 ng; 35614, Addgene), gag-Cas9 (2,250 ng; 181752, Addgene), and each sgRNA (8,800 ng) selected in Example 1 were mixed in polyethylenimine (120 μg; 23966-100, Polyscience Inc.) and Opti-MEM medium (8 ml; 31985-070, Thermo Fisher Scientific). After removing 6 ml of the DMEM medium from the culture, the prepared Opti-MEM mixture was added to perform transfection. After 24 hours, the culture medium was completely removed and replaced with fresh DMEM medium. After 48 hours from the transfection, the supernatant was collected and centrifuged at 500 g for 5 minutes to remove cell debris. Subsequently, the supernatant was filtered using a 0.45 μm CA filter (S6555-FMOSK, Sartorius). 5×PEG-it Virus Precipitation Solution (System Biosciences, LV825A-1) was added to precipitate eVLPs, followed by incubation at 4° C. overnight. The following day, the supernatant was centrifuged at 1,500 g for 30 min at 4° C. to pellet the eVLPs. The resulting pellets were resuspended and concentrated in 1×HIV-safe Manager (LGV-1022B, Lugen SCI). The concentrated eVLPs were stored at −80° C. and thawed on ice immediately before use. To maintain consistency between experiments, all eVLPs used in cell culture studies were concentrated using this standardized method. This concentration process provides a yield of approximately 6 to 6.5×10⁸ eVLPs/μl.

To compare the NUTM1 editing effects, the CCR5 (C-C chemokine receptor type 5) gene was selected as a control, and eVLPs were produced in the same manner using an sgRNA targeting CCR5 (Sequence: GGTGACAAGTGTGATCACTTGGG (SEQ ID NO: 13), PAM: TGG).

### Example 3: Editing of NUT Cell Lines Using CRISPR/Cas9 System

The effect of the CRISPR-Cas9 system on inhibition of NUT protein expression was confirmed using HCC2429, 10-15, or JCM1, which are NUT carcinoma cell lines expressing the NUT protein. HCC2429 cells were cultured in RPMI1640 (SH30027.01, Hyclone) medium, and 10-15 and JCM1 cells were cultured in Dulbecco's modified Eagle's medium (DMEM, SH30243.01, Hyclone) supplemented with 10 mg/ml antibiotics (penicillin and streptomycin, P/S, Gibco) and 10% fetal bovine serum (FBS, Life Technologies) in an incubator with 5% CO₂ at 37° C.

The NUT carcinoma cell lines were cultured on plates suitable for the experimental purpose, and the following day, each cell line was transfected with each Cas9-eVLP produced in Example 2. At this time, Cas9-eVLPs containing sg E6 and Cas9-eVLPs containing sg E8-1v2 were mixed in a ratio of 1:1 and co-transfected (hereinafter labeled as sg E6C or E6C). After 24 hours, the medium was replaced with fresh medium, and after 72 hours, cover glasses or cells were harvested depending on the purpose. For H&E staining and immunofluorescence experiments, the NUT carcinoma cell lines were cultured in 12-well plates containing cover glasses, and cover glasses were ultimately harvested. In contrast, for other experiments requiring NUTM1-edited NUT carcinoma cell lines, cells were cultured in 24-well plates, and the cells were finally harvested.

### Example 4: H&E Staining

The cover glasses harvested in Example 3 were fixed with 4% paraformaldehyde for 10 minutes. After washing the cover glasses three times with PBS, staining was performed in the following order: once with water for 10 seconds, three times with hematoxylin for 10 seconds each, twice with water for 10 seconds each, once with eosin solution for 10 seconds, twice with 90% ethanol for 10 seconds each, three times with 100% ethanol for 10 seconds each, and three times with xylene for 10 seconds each. Thereafter, a drop of xylene and Canada balsam was applied to a slide, and the slide was covered with the cover glass facing downward. The stained slides were dried and then scanned using an Aperio Scanscope AT2 (Leica Biosystems).

### Example 5: Analysis of Differentially Expressed Genes

Analysis and visualization of differentially expressed genes using transcriptome sequencing data from NUT carcinoma cell lines were performed using a publicly available analysis tool. Differences in the expression of each gene were compared using adjusted P-values and log FC. It can be interpreted that the greater the dispersion shown in the graph, the more significant the differences in expression levels.

### Example 6: Quantitative PCR (qPCR)

qRT-PCR analysis was performed to confirm the increase in TACSTD2 expression levels in HCC2429 and JCM1 cell lines. Specifically, RNA was extracted and quantified from the transfected cells harvested in Example 3 using an RNeasy Mini Kit (Qiagen, #74106) according to the manufacturer's instructions. CDNA was synthesized from 1 μg of total RNA using SuperScript™ IV Reverse Transcriptase (Thermo Fisher Scientific, 18090200). qRT-PCR was performed in a 384-well plate using a PRISM® 7900HT Fast Real-Time PCR System (Applied Biosystems) or a QuantStudio™ 6 Flex Real-Time PCR System (Applied Biosystems) with 2×SYBR Green Master Mix (Applied Biosystems, #4368702). Gene expression was normalized to the housekeeping gene GUSB (glucuronidase beta). All experiments were performed in triplicate for each sample, and statistical analysis was performed using a t-test.

### Example 7: Western Blot Assay

Intracellular protein expression levels were confirmed by Western blot assay. Specifically, the cells harvested in Example 3 were lysed with RIPA buffer (Thermo Fisher) containing a protease inhibitor, and then centrifuged at 13,000 rpm and 4° C. for 25 minutes to obtain the supernatant. The protein amount was quantified using the BSA or Bradford assay. Protein lysates were separated on SDS-PAGE gels and transferred to PVDF membranes (Millipore), and then immunoblotted with NUTM1 (C52B1) antibody (1:500 dilution; Cell Signaling Technologies, #3625), TACSTD2 antibody (1:2000 dilution; Atlas Antibodies, #HPA055067), and Beta Actin (C4) antibody (Santa Cruz Biotechnology, sc-47778).

### Example 8: Flow Cytometry

Flow cytometry was performed to analyze Trop2 expression in NUT carcinoma cells subjected to NUTM1 targeted editing. Specifically, the cells harvested in Example 3 were dissociated using Accutase (Thermo Fisher Scientific, 00-4555-56). The dissociated cells were centrifuged and then resuspended in FACS buffer (PBS containing 1% FBS) at a concentration of 2 to 3×10⁶ cells/mL. The prepared cells were incubated with a PE anti-human TACSTD2 (TROP2) antibody (Biolegend, 363804) in PBS containing 1% FBS at 4° C. for 1 hour. After washing the cells twice, they were transferred to FACS tubes (Falcon, #352235), and flow cytometry analysis was performed using a BD FACS Lyric instrument.

### Example 9: Analysis of Genome Editing Ratio

To confirm the indel frequency in cells by the CRISPR-Cas9 system, genomic DNA was extracted from the cells harvested in Example 3 using a DNA Mini Kit (Qiagen, #51306) according to the manufacturer's instructions. To determine the indel frequency in the target DNA regions, the target genomic regions were amplified by PCR using Accupower Hotstart PCR Premix (Bioneer, #K5051-1). A portion of the PCR amplicons was purified using a PCR Purification Kit (Qiagen, 28106) and used for Sanger sequencing. Analysis of the Sanger sequencing results was performed using a publicly available analysis tool.

### Example 10: Immunofluorescence Analysis

The cover glasses harvested in Example 3 were fixed with 4% paraformaldehyde at a depth of 2 to 3 mm at room temperature for 15 minutes, followed by washing three times with PBS for 5 minutes each. The resulting cover glasses were treated with a Blocking Buffer (5% Skim Milk (232100, BD) in 1×TBST (T2007-100-74, BIOSESANG)) for 60 minutes to block non-specific binding. After removing the Blocking Buffer, the diluted primary antibodies (NUT antibody (Cell Signaling Technology, 3625S) and TACSTD2 (TROP2) antibody (Thermo Fisher Scientific, 53-6024-82)) were incubated overnight at 4° C. The following day, the cells were washed three times with PBS for 5 minutes each, and fluorophore-conjugated secondary antibodies (Thermo Fisher Scientific, A-11070 or A-11011) diluted in antibody dilution buffer were incubated for 1 to 2 hours while protected from light. After the incubation was complete, the cells were washed three times with PBS for 5 minutes each while protected from light. Digital images were captured using an LSM 700 or LSM 780 ZEISS laser scanning confocal microscope (Carl Zeiss). The image data were processed using the integrated LSM software.

### Example 11: Cytotoxicity Analysis

Drug-dependent cytotoxicity was analyzed. 293 FT (Invitrogen, R70007) or HCC2429 cell lines were used, and depending on the characteristics of the cell lines, 500 to 5,000 cells per well were seeded in 100 μl of medium in a 96-well plate. The treated drugs included sacituzumab (in-house, Abion), IFN-β mutein (Abion), sacituzumab-IFN-β mutein fusion protein (Abion), and a control mAb (non-TROP2-targeting antibody, anti-botulinum toxin antibody, in-house, Abion). These were treated at concentrations ranging from 10⁻⁶ to 10³ nM depending on the type. After 72 to 120 hours, the culture medium in each well was replaced with a mixture of 10 μL of Ez-Cytox (DoGenBio, #EZ-3000) and 90 μL of fresh medium. The plates were incubated in a humidified 37° C. incubator containing 5% CO₂ for 2 to 4 hours, and absorbance was measured at 450 nm using a SpectraMax® 96-well plate reader (Molecular Devices). Based on the measured values, IC₅₀ values were calculated using a publicly available analysis tool.

### Example 12: Cell Proliferation Analysis

The NUTM1-edited cells harvested in Example 3 were seeded in a 96-well plate at 3×10³ cells per well and then treated with representative TROP2-targeted anticancer agents. The cells were cultured for 72 hours with 10-fold serially diluted Trop2 antibody (sacituzumab), sacituzumab-IFN-β mutein fusion protein (Abion), or a control mAb-IFN-β mutein fusion protein (Abion) using a non-TROP2-targeting antibody. Subsequently, the culture medium in each well was replaced with a mixture of 10 μL of Ez-Cytox (DoGenBio, #EZ-3000) and 90 μL of fresh medium, and the cells were incubated in an incubator for 3 hours. The change in cell proliferation rate was calculated by measuring the absorbance at 450 nm.

### Example 13: Antibody-Dependent Cellular Cytotoxicity (ADCC) Assay

Using NK-92 MI-CD16a cells, which stably express CD16a, as effector cells, an antibody-dependent cellular cytotoxicity assay was performed on the NUTM1-edited HCC2429 cell line harvested in Example 3 against the TROP2-targeted anticancer agents treated in Example 12. HCC2429 cells were seeded in a 96-well plate at 2×10⁴ cells per well and cultured overnight, followed by further incubation for 4 hours in an incubator with 2×10⁴ effector cells and various concentrations of drugs. HCC2429 cell lysis was calculated by measuring lactate dehydrogenase release using the CytoTox 96® Non-Radioactive Cytotoxicity Assay Kit (G1780, Promega).

### Experimental Example 1: Effect of NUTM1 Gene Editing

The present inventors observed that when differentiation was induced in NUT carcinoma cell lines through NUTM1 gene editing using a CRISPR/Cas9 system containing NUTM1-targeting sgRNAs, growth inhibitory effects were exhibited in the cells, but cell death was not achieved, and differentiation into squamous type cells occurred (FIG. 2A). That is, as shown in the schematic diagram in FIG. 2A, NUTM1 gene editing shows that characteristic changes occur in NUT carcinoma cell lines, such as an increase in cell size and cytoplasmic volume, and differentiation of the cells into flat, squamous-like cells.

Specific experimental results show that, in various NUT carcinoma cell lines (HCC2429, 10-15, and JCM1), differentiation into a squamous-like morphology was observed in the experimental groups labeled as sg E3-2 (SEQ ID NO: 2), sg E3-9 (SEQ ID NO: 9), and E6C (a mixture of sg E6 (SEQ ID NO: 10) and sg E8-1v2 (SEQ ID NO: 12) in a ratio of 1:1 in the form of eVLPs), compared to the control group in which the CCR5 gene was edited (FIG. 2B).

Moreover, differentially expressed genes were visualized using volcano plots through transcriptome analysis of the control group (CCR5 edited-NUT cell lines) and the NUTM1 edited-NUT cell lines. As a result, it was interestingly confirmed that the expression of the TROP2 protein, encoded by the TACSTD2 gene, was increased in the NUTM1-edited NUT cell lines (FIG. 2C).

Accordingly, as a result of analyzing the relationship between the degree of cancer cell differentiation and TROP2 protein expression in NC patient tissues, it was discovered for the first time that there is a positive correlation between the two variables and that patients exhibiting a high degree of NC cell differentiation and TROP2 positivity are associated with a relatively better prognosis.

### Experimental Example 2: Determination of Increased Trop2 Expression in NUTM1-Edited NUT Carcinoma Cell Lines by NUTM1 Gene Editing

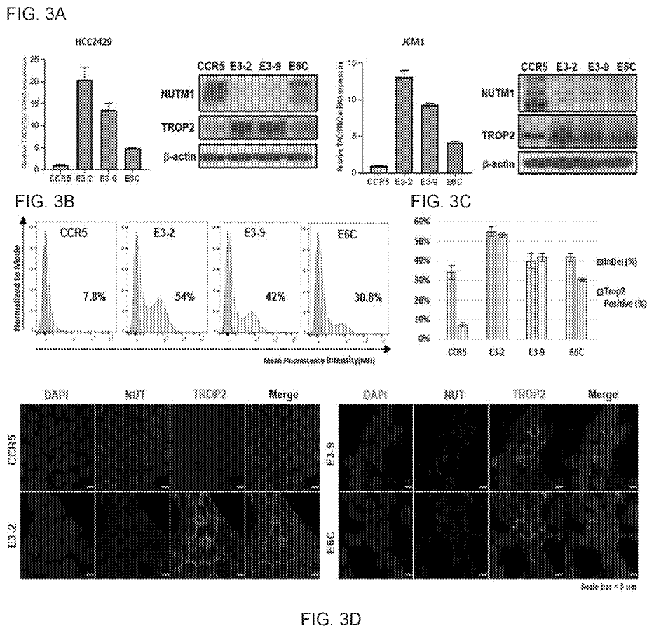

The present inventors verified the increase in Trop2 expression induced by NUTM1 gene editing through qPCR and Western blot. As a result, as shown in FIG. 3A, TACSTD2 mRNA expression was significantly increased by approximately 20-fold in HCC2429 cells and approximately 13-fold in JCM1 cells, compared to the CCR5 control group. Western blot analysis confirmed that NUTM1 gene editing was successfully performed in each experimental group, resulting in increased Trop2 protein expression.

Furthermore, the proportion of cell lines with increased Trop2 expression was verified by flow cytometry. As a result, as shown in FIG. 3B, Trop2 expression was observed in only 7.8% of cells in the CCR5 control group; however, the proportion of cells showing TROP2 expression was significantly increased to 54%, 42%, and 30.8% in the experimental groups including sg E3-2 (SEQ ID NO: 2), sg E3-9 (SEQ ID NO: 9), and E6C (a mixture of sg E6 (SEQ ID NO: 10) and sg E8-1v2 (SEQ ID NO: 12) in a ratio of 1:1 in the form of eVLPs), respectively.

In addition, to verify the indel frequency in cells by CRISPR-Cas9, the InDel rate in the NUTM1 gene and the Trop2 expression rate in the transfected NUT carcinoma cell lines were compared. As a result, as shown in FIG. 3C, it was confirmed that Trop2 expression levels were increased in almost all NUT carcinoma cell lines in which the NUTM1 gene was edited.

Additionally, the decrease in NUTM1 expression and the increase in Trop2 expression were confirmed by immunofluorescence imaging. As a result, as shown in FIG. 3D, NUTM1 gene editing induced the loss of the characteristic intranuclear speckled pattern of NUT carcinoma and increased TROP2 expression on the cell membrane. In particular, it was confirmed through green fluorescence that TROP2 expression was most significantly increased in the sg E3-2 (SEQ ID NO: 2) experimental group.

### Experimental Example 3: Determination of Synergistic Antitumor Effect of TROP2-Targeting ACFP or ADC and NUTM1 Inhibitor in NUT Carcinoma Cells

Based on the results in Experimental Examples 1 and 2, the present inventors established a strategy of: 1) recommending a TROP2-targeted therapeutic agent for patients with a high degree of NUT cell differentiation and TROP2-positivity; and 2) inducing cancer cell differentiation and TROP2 expression through NUTM1 gene editing to enable subsequent TROP2-targeted therapy.

Moreover, as a strategy of targeted therapy, the present inventors evaluated the efficacy of sacituzumab-IFN-β mutein fusion protein (ACFP) and sacituzumab govitecan (ADC), which are representative drugs that can be used by binding to the Trop2-antibody (sacituzumab).

At this time, HCC2429 NUT carcinoma cells expressing the BRD4-NUTM1 fusion gene were used as a positive control, and 293 FT cells, which do not express the BRD4-NUTM1 fusion gene, were used as a negative control.

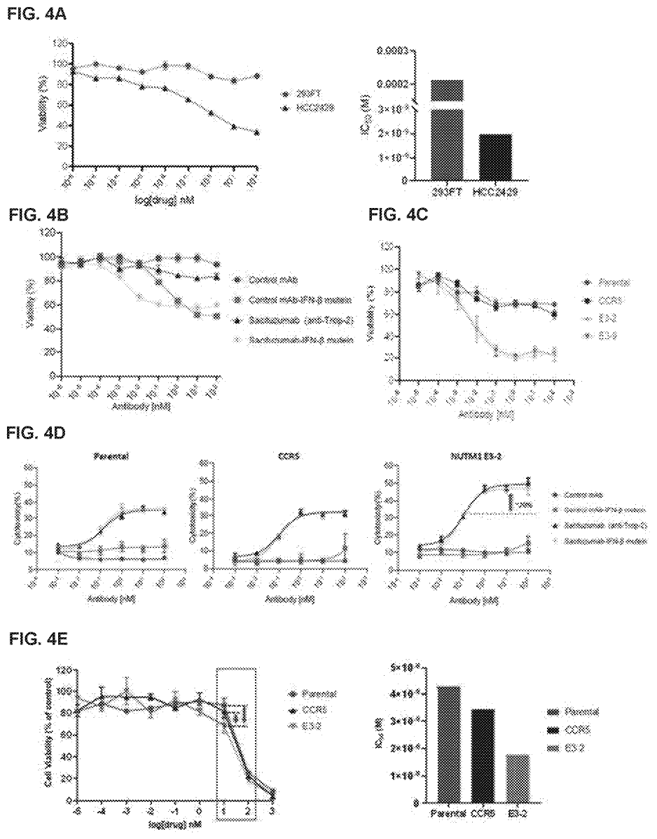

As a result, as shown in FIG. 4A, IFN-β mutein exhibited NUT carcinoma-specific cytotoxicity, decreasing the viability of HCC2429 cells, a cell line expressing the BRD4-NUTM1 fusion gene, while no cytotoxicity was observed in normal cells (293 FT). In addition, as shown in FIG. 4B, when treated with sacituzumab-IFN-β mutein fusion protein (ACFP, provided by Abion), which is an IFN-β mutein fused with the Trop2-antibody (sacituzumab), the same NUT carcinoma cell-specific cytotoxicity was observed.

Accordingly, using the CRISPR/Cas9 system containing NUTM1-targeting sgRNAs (sg E3-2 and sg E3-9), the present inventors treated each cell in which the NUTM1 gene was edited with sacituzumab or sacituzumab-IFN-β mutein fusion protein (ACFP), respectively.

As a result, as shown in FIG. 4C, when HCC2429 cells (labeled as sg E3-2 and sg E3-9) in which Trop2 protein expression was increased by editing NUTM1 were treated with sacituzumab-IFN-β mutein fusion protein (ACFP), a decrease in the viability of the HCC2429 cells was observed. That is, an antitumor synergistic effect between NUTM1 gene editing and the TROP2-targeted anticancer agent was confirmed. As controls, CCR5-edited HCC2429 cells or parental HCC2429 cell lines in which no gene editing was performed were used.

Furthermore, as shown in FIG. 4D, when NUTM1-edited cells (sg E3-2-treated group) were treated with sacituzumab or sacituzumab-IFN-β mutein fusion protein (ACFP) in combination with NK-92 MI-CD16a cells, it was confirmed that the antibody-dependent cellular cytotoxicity (ADCC) effect by the NK cell line was significantly increased compared to the parental and CCR5 controls. As controls, CCR5-edited HCC2429 cells or parental HCC2429 cell lines in which no gene editing was performed were used.

In addition, as shown in FIG. 4E, when HCC2429 cells (sg E3-2-treated group) in which Trop2 protein expression was increased by editing NUTM1 were treated with sacituzumab govitecan (Trodelvy), which is an ADC, it was confirmed that the drug responsiveness was increased compared to the parental and CCR5 controls. As controls, CCR5-edited HCC2429 cells or parental HCC2429 cell lines in which no gene editing was performed were used.

### Experimental Example 4: Determination of Trop2 Expression Levels in Patient Tissues According to the Degree of NC Differentiation

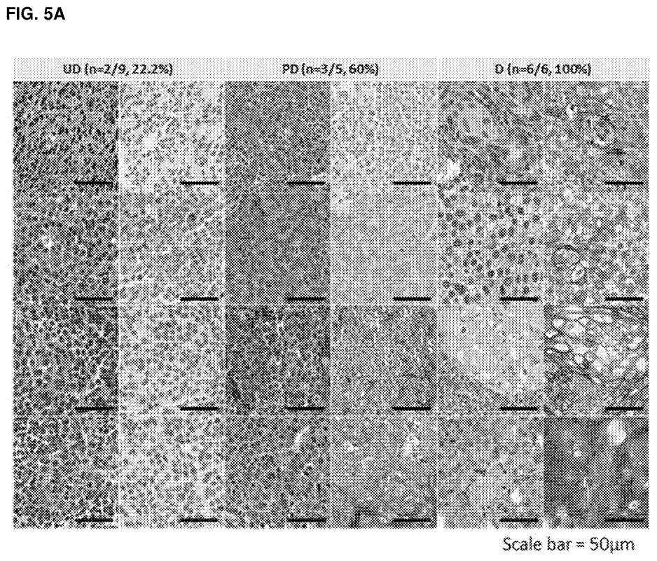

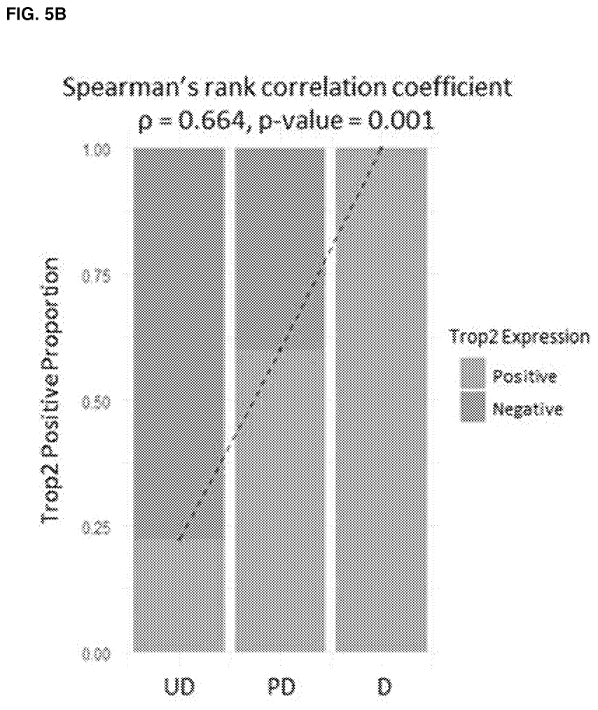

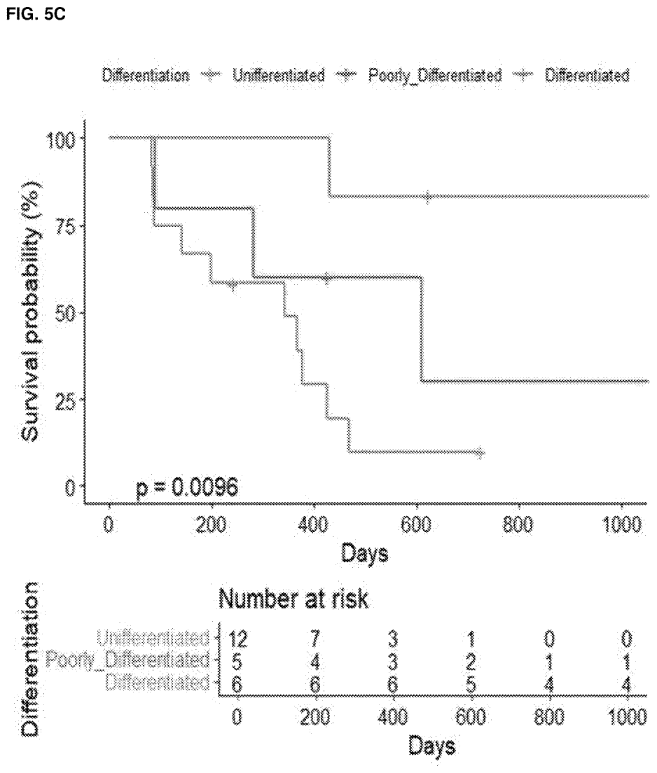

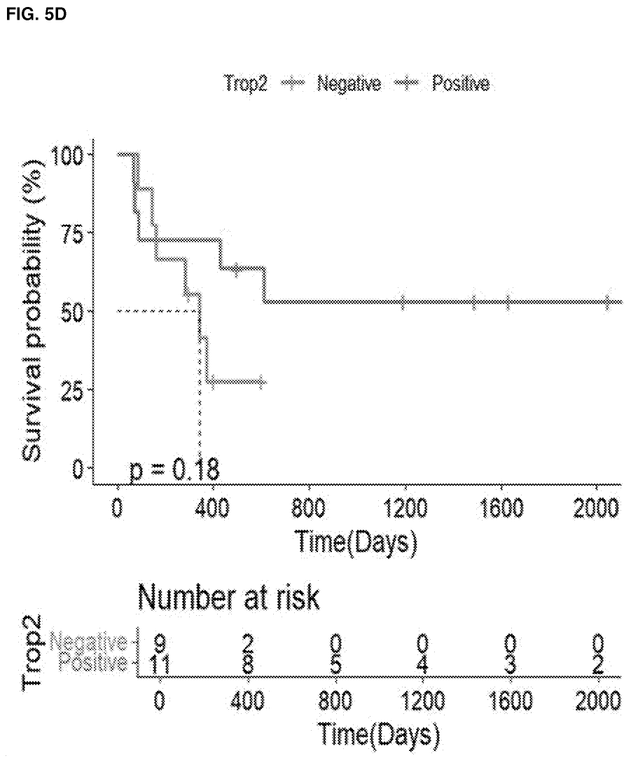

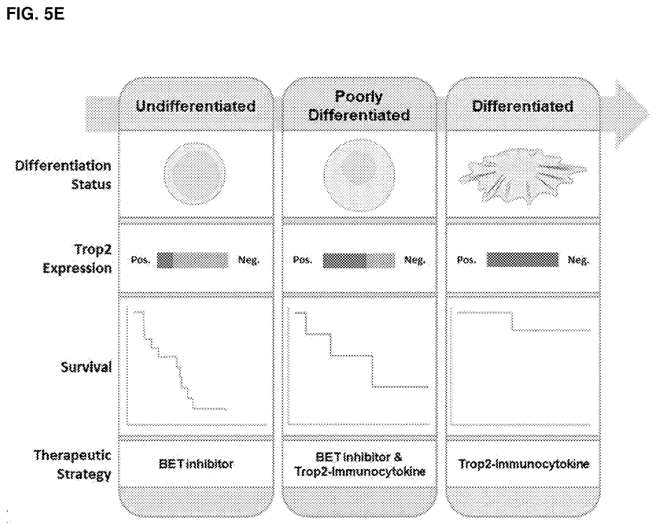

In order to analyze the relationship between the degree of NUT cell differentiation and TROP2 protein expression in NC patient tissues, the present inventors classified NC patients histologically according to the degree of differentiation and then matched the HE images and TROP2 IHC (immunohistochemistry) images obtained by the methods described in Examples 4 and 10 (FIG. 5A).

As a result, as shown in FIG. 5B, the present inventors discovered that there is a positive correlation between the two variables, i.e., the degree of NUT cell differentiation and TROP2 protein expression, and proved that the two variables show a high positive correlation by confirming that the Spearman's rank correlation coefficient (p) value is 0.664 through statistical calculations (p-value=0.001).

Moreover, as shown in FIG. 5C, the present inventors analyzed the overall survival of NC patients according to the degree of NUT cell differentiation using a Kaplan-Meier survival analysis (Kaplan-Meier estimate) graph, and as a result, confirmed that a better prognosis was observed in patients with a higher degree of NUT cell differentiation (p-value=0.0096).

Furthermore, as shown in FIG. 5D, the present inventors analyzed the overall survival of NC patients according to TROP2 protein expression using a Kaplan-Meier survival analysis graph, and as a result, confirmed a tendency for a better prognosis in TROP2-positive patients compared to TROP2-negative patients (p-value=0.18).

Taken together, the present inventors discovered that the expression level of Trop2 increased when the differentiation of NUT cells was induced through NUTM1 gene editing using NUTM1-targeting sgRNAs and the CRISPR/Cas9 system in NC cell lines. Moreover, as a result of analyzing the relationship between the degree of NUT cell differentiation and TROP2 protein expression in NC patient tissues, it was discovered for the first time that there is a positive correlation between the two variables and that patients exhibiting a high degree of NUT cell differentiation and TROP2 positivity are associated with a relatively better prognosis.

Based on these findings, as shown in FIG. 5E, the present invention demonstrated the characteristics and treatment strategies for NC carcinoma according to the degree of NUT cell differentiation described above; that is, the present invention proved that an advantageous therapeutic effect can be exhibited when a TROP2-targeted therapeutic is applied to TROP2-positive patients with a high degree of NUT cell differentiation. In addition, conventional BET inhibitors targeting the mechanism of NC may be considered for TROP2-negative patients, but there is a limit to their practical use due to high side effects. Likewise, according to the strategy of the present invention, in patients with a low degree of NUT cell differentiation, TROP2-targeted therapy becomes possible by inducing NUT cell differentiation and TROP2 expression through NUTM1 gene editing, thereby sensitizing NUT carcinoma to drugs. As an effective and novel drug therapy, the present invention will realize personalized medicine, effectively improve patient survival rates through targeted therapy, and contribute to enhancing the quality of life for patients by reducing unnecessary anticancer treatments.

While specific aspects of the present invention have been described in detail above, it will be apparent to those skilled in the art that such specific descriptions are merely preferred embodiments and that the scope of the present invention is not limited thereby. Accordingly, the substantial scope of the present invention will be defined by the appended claims and their equivalents.

1. A pharmaceutical composition for preventing or treating cancer, comprising an NUT midline carcinoma family member 1 (NUTM1) inhibitor and a tumor-specific antigen-targeted anticancer agent as active ingredients.

2. The composition according to claim 1, wherein the NUTM1 inhibitor is at least one selected from the group consisting of a CRISPR/Cas system comprising a guide RNA (gRNA) targeting the NUTM1 gene, an antisense oligonucleotide (ASO), a small interference RNA (siRNA), a short hairpin RNA (shRNA), a microRNA (miRNA), and a ribozyme.

3. The composition according to claim 2, wherein the guide RNA targeting the NUTM1 gene is a guide RNA consisting of a nucleotide sequence selected from the group consisting of SEQ ID NOs: 1 to 12.

4. The composition according to claim 1, wherein the NUTM1 inhibitor is at least one selected from the group consisting of a compound, a peptide, a peptidomimetic, a substrate analog, an aptamer, and an antibody, each of which specifically binds to the NUT protein.

5. The composition according to claim 1, wherein the NUTM1 inhibitor increases the expression level or activity of at least one tumor-specific antigen selected from the group consisting of trophoblast cell surface antigen 2 (Trop2), epidermal growth factor receptor (EGFR), vascular endothelial growth factor receptor (VEGFR), CD20, CD38, RANK-L, BTK, Bcr-abl, PDGFR/FGFR families, MEK/RAF, HER2/Neu, ubiquitin, JAK, MAP2K, ALK, PARP, transforming growth factor β (TGFβ) receptor, proteasome, Bcl-2, C-Met, VR1, VR2, VR3, c-kit, AXL, RET, Braf, DNA methyltransferase (DNMT), CDK4/6, and STING.

6. The composition according to claim 1, wherein the tumor-specific antigen-targeted anticancer agent is a targeted anticancer agent that targets at least one tumor-specific antigen selected from the group consisting of trophoblast cell surface antigen 2 (Trop2), epidermal growth factor receptor (EGFR), vascular endothelial growth factor receptor (VEGFR), CD20, CD38, RANK-L, BTK, Bcr-abl, PDGFR/FGFR families, MEK/RAF, HER2/Neu, ubiquitin, JAK, MAP2K, ALK, PARP, transforming growth factor β (TGFβ) receptor, proteasome, Bcl-2, C-Met, VR1, VR2, VR3, c-kit, AXL, RET, Braf, DNA methyltransferase (DNMT), CDK4/6, and STING.

7. The composition according to claim 6, wherein the targeted anticancer agent targeting the TROP2 tumor-specific antigen is at least one selected from the group consisting of an anti-Trop2 antibody specifically binding to a Trop2 protein, an anti-Trop2 antibody-drug conjugate (ADC), an anti-Trop2 antibody-cytokine fusion protein (ACFP), a peptide, a peptidomimetic, a substrate analog, an aptamer, and a compound.

8. The composition according to claim 6, wherein the targeted anticancer agent targeting the TROP2 tumor-specific antigen is at least one selected from the group consisting of sacituzumab, a sacituzumab-IFN-β mutein fusion protein, sacituzumab govitecan (Trodelvy), datopotamab deruxtecan (DS-1062), ESG-401, SKB-264, DAC-02, and BAT-8003.

9. The composition according to claim 1, wherein the cancer is a BRD4-NUTM1-positive cancer.

10. The composition according to claim 9, wherein the cancer is at least one selected from the group consisting of NUT carcinoma, lung cancer, thymic cancer, and renal cancer.

11. The composition according to claim 10, wherein the NUT carcinoma is in an undifferentiated or differentiated state.

12. (canceled)

13. An adjuvant composition for a tumor-specific antigen-targeted anticancer agent, comprising an NUTM1 inhibitor as an active ingredient.

14. The composition according to claim 13, wherein the composition is used separately, sequentially, or simultaneously with the tumor-specific antigen-targeted anticancer agent.

15-16. (canceled)

17. A method for treating NUT carcinoma in a subject, comprising:
(a) evaluating TROP2 expression levels or cellular differentiation status in cancer cells derived from the subject; and
(b) based on the evaluation results,
(i) administering a TROP2-targeted anticancer agent to the subject when the cancer cells are highly differentiated or TROP2-positive; or
(ii) inhibiting NUTM1 expression or function to induce differentiation and TROP2 expression in the cancer cells when the cancer cells are poorly differentiated or TROP2-negative and subsequently administering a TROP2-targeted anticancer agent to the subject.

18. The method of claim 17, wherein the NUTM1 inhibitor is selected from the group consisting of a CRISPR/Cas system comprising a guide RNA (gRNA) targeting the NUTM1 gene, an antisense oligonucleotide (ASO), a small interfering RNA (siRNA), a short hairpin RNA (shRNA), a microRNA (miRNA), and a ribozyme.

19. The method of claim 18, wherein the guide RNA targeting the NUTM1 gene comprises a nucleotide sequence selected from SEQ ID NOs: 1 to 12.

20. The method of claim 17, wherein the NUTM1 inhibitor is selected from the group consisting of a compound, peptide, peptidomimetic, substrate analog, aptamer, and antibody, each of which specifically binds to the NUT protein.

21. The method of claim 17, wherein the TROP2-targeted anticancer agent is selected from the group consisting of an anti-TROP2 antibody, an anti-TROP2 antibody-drug conjugate (ADC), an anti-TROP2 antibody-cytokine fusion protein (ACFP), a peptide, a peptidomimetic, a substrate analog, an aptamer, and a compound.

22. The method of claim 17, wherein the TROP2-targeted anticancer agent is selected from the group consisting of sacituzumab, a sacituzumab-IFN-β mutein fusion protein, sacituzumab govitecan (Trodelvy), datopotamab deruxtecan (DS-1062), ESG-401, SKB-264, DAC-02, and BAT-8003.
